(19) United States
(12) Patent Application Publication
(10) Pub. No.: US 2025/0128085 A1
(43) Pub. Date: Apr. 24, 2025

(54) LIGHT THERAPY HEADPHONES

(71) Applicant: Ava RUMMLER, Moscow, ID (US)

(72) Inventor: Ava RUMMLER, Moscow, ID (US)

(21) Appl. No.: 18/489,580

(22) Filed: Oct. 18, 2023

(51) Int. Cl. A61N 5/06; H04R 1/10

(52) U.S. Cl. CPC A61N 5/0616; H04R 1/1008; H04R 1/105; A61N 2005/0647; A61N 2005/0652; A61N 2005/0661

(57) ABSTRACT

Disclosed embodiments include headphones, or other headsets, that include ultra-violet B (UVB) (e.g., 290 nm and 315 nm) light emitting diodes (LEDs) or other light sources to provide a source of vitamin D while worn by a wearer.

### FIELD OF THE DISCLOSURE

This disclosure relates generally to headphones, or other headsets, meant to be worn on a person's head. More particularly, this disclosure relates to headphones, or other headsets, that include light emitting diodes (LEDs) or other light sources to provide a source of vitamin D.

### BACKGROUND

A lack of vitamin D is a leading cause to anxiety and depression to lots of people such as pilots and others that are not often outside where the sun provides vitamin D. For example, according to a journal posted by BMC, BioMedCentral.com, 13.5% of pilots met the “depression threshold-PHQ-9 total score ≥10” in a study conducted by Harvard's Chan School of Public Health. This is more than the national average of 9.5% and includes people under all demographics over the age of 18. Pilots are often isolated from being outside for up to two weeks at a time which prevents them from absorbing the proper amount of vitamin D from the sun and can lead to unhealthy mental states. Likewise, others that have primarily indoor existences or otherwise do not get exposure to vitamin D may suffer similarly.

Therefore, a preventative to depression is vitamin D absorption. Vitamin D can come in the form of non-natural light therapy, an artificial technique for one to absorb vitamin D. Many of these artificial techniques come in the form of lamps, masks, and medical treatments. These options have been deemed successful, but only when consistently used for sufficient periods of time. The needed consistency and exposure time may be difficult for some, such as pilots who spend up to 13 hours flying at a time, which means that normal artificial vitamin D treatments can't be utilized for that time. Other drawbacks, inefficiencies, and issues with current systems and methods also exist.

### SUMMARY

Accordingly, disclosed embodiments address the above, and other drawbacks, inefficiencies, and issues with existing systems and methods. An embodiment of one solution to pilots' inability to perform accessible artificial vitamin D therapy is to implement the artificial vitamin D source into the headsets that pilots typically wear during an entire flight (e.g., from pre boarding to landing).

Disclosed embodiments include headgear to be worn on a wearer's head, the headgear including an earcup, and a UVB light source within the earcup. In some embodiments the UVB light source comprises a light emitting diode (LED). In some embodiments the UVB light source comprises a strip of LEDs. In some embodiments the earcup further comprises a cushion around a perimeter of the earcup and the strip of LEDs is positioned within a space on an inner surface of the cushion. In some embodiments the earcup further comprises a space for a wearer's ear within the earcup and the LED is positioned within the space for a wearer's ear. In some embodiments the UVB light source comprises at least one LED and the UVB light source substantially fills the space for a wearer's ear. In some embodiments the UVB light source comprises at least one LED and the UVB light source is cross shaped.

In some embodiments the earcup includes a sound emitting source and a cushion and the UVB light source is positioned within a space between the cushion and the sound emitting source. In some embodiments the earcup includes a sound emitting source and the UVB light source is positioned on a front surface of the sound emitting source. In some embodiments the UVB light source comprises at least one LED and the UVB light source is cross shaped.

Disclosed embodiments also include headgear to be worn on a wearer's head, the headgear including at least one earcup, a headband to be worn over a wearer's head, and a UVB light source positioned in the headband. Other embodiments also exist.

### BRIEF DESCRIPTION OF THE DRAWINGS

FIG. 1 is a schematic illustration of an embodiment of headgear in accordance with the disclosure.

FIG. 2 is a schematic illustration of the inside of an earcup in accordance with disclosed embodiments.

FIG. 3 is a schematic illustration of the inside of an earcup in accordance with disclosed embodiments.

FIG. 4 is a schematic illustration of the inside of an earcup in accordance with disclosed embodiments.

FIG. 5 is a schematic illustration of the inside of an earcup in accordance with disclosed embodiments.

FIG. 6 is an embodiment of headgear in accordance with the disclosure.

While the disclosure is susceptible to various modifications and alternative forms, specific embodiments have been shown by way of example in the drawings and will be described in detail herein. However, it should be understood that the disclosure is not intended to be limited to the particular forms disclosed. Rather, the intention is to cover all modifications, equivalents and alternatives falling within the spirit and scope of the invention as defined by the appended claims.

### DETAILED DESCRIPTION

Sunlight, in particular ultra-violet B (UVB) between the wavelengths of 290 nm and 315 nm, is the main source for producing vitamin D in the skin and is the primary source of vitamin D for the body. As disclosed herein, UVB LEDs may also be used to provide light onto skin and provide vitamin D. LEDs are particularly useful because of, among other things, their relatively small size and power requirements, which allow them to be incorporated into existing headgear hardware systems with relative ease. While LEDs are preferable for the foregoing, and other, reasons other light sources that emit in the wavelengths of 290 nm and 315 nm may also be used in accordance with the embodiments disclosed herein.

As used herein, “headgear,” “headset,” and “headphones,” all refer to devices meant to be worn over or in a wearer's ears and may be for functions such as noise cancellation, hearing protection, listening to music or other sound sources, communicating with others (such as air traffic control and pilot headsets, walkie-talkies, etc.), receiving and making telephone calls, providing warmth, and the like. Headgear may be wired or wireless as is known in the art.

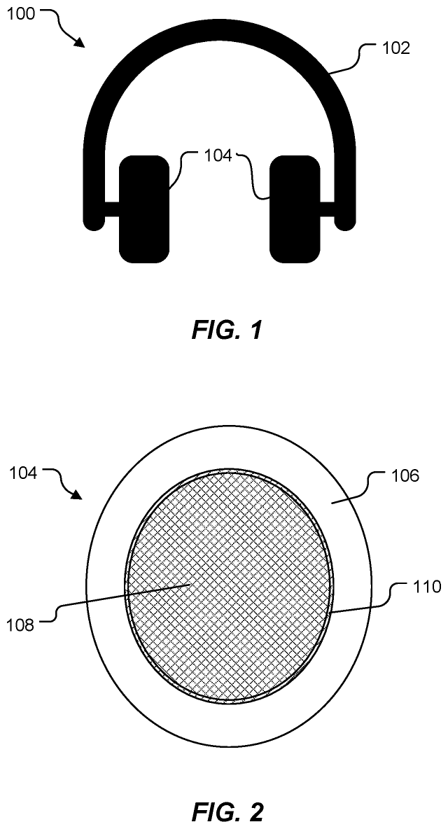

FIG. 1 is a schematic illustration of an embodiment of headgear 100 in accordance with the disclosure. As shown, headgear 100 may include a headband 102 that connects one or two earcups 104. Some embodiments of headgear 100 may also include a microphone (not shown) for communication of the wearer's voice. Depending upon the intended application of the headgear 100 (e.g., hearing protection, communication, listening to music, etc.) the style, shape, size, thickness, and other features and attributes of the headband 102 and earcups 104 may vary.

FIG. 2 is a schematic illustration of the inside (i.e., wearer's ear side) of an earcup 104 in accordance with disclosed embodiments. As shown embodiments of earcup 104 may include a pad or cushion 106 that covers a wearer's ear and provides comfort and a sound emitter or speaker 108. A UVB light source 110 may be included in a space between the cushion 106 and the speaker 108 as shown. In this embodiment, the UVB light source 110 may be LEDs on a strip that encircles the earcup 104 as shown. The power source for the UVB light source 110 may be provided through the headgear 100 wiring (for wired embodiments) or through connection to a battery (for wireless embodiments).

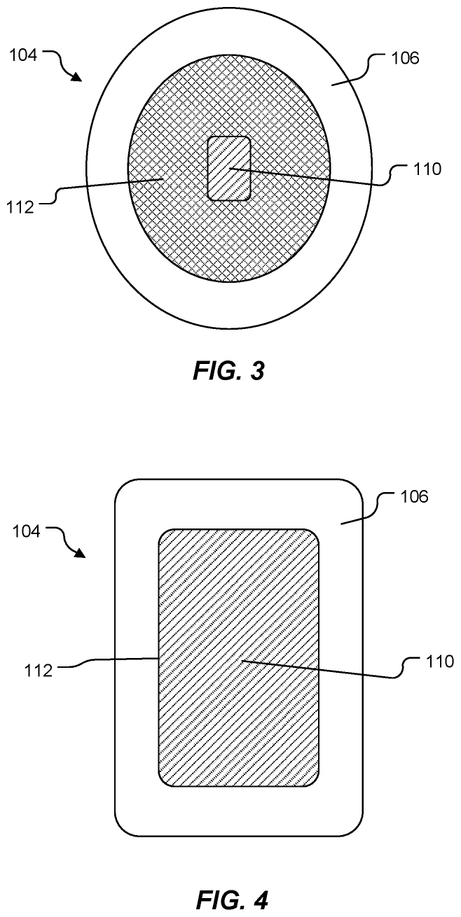

As would be understood by those of skill in the art having the benefit of this disclosure, for embodiments of headgear 100 that do not produce sound (e.g., hearing protection or warmth providing) speaker 108 may not be included and a cavity or space 112 may be included in that location instead. FIG. 3 is a schematic illustration of an earcup 104 having a space 112 for a wearer's ear. As also shown a UVB light source 110 may be included in that space 112.

FIG. 4 is a schematic illustration of the inside of an earcup 104 in accordance with disclosed embodiments. As shown in this embodiment, the shape of the earcup 104 may vary and be generally rectangular or otherwise shaped. As also shown UVB light source 110 may substantially fill the space 112 in the earcup 104 adjacent to the wearer's ear.

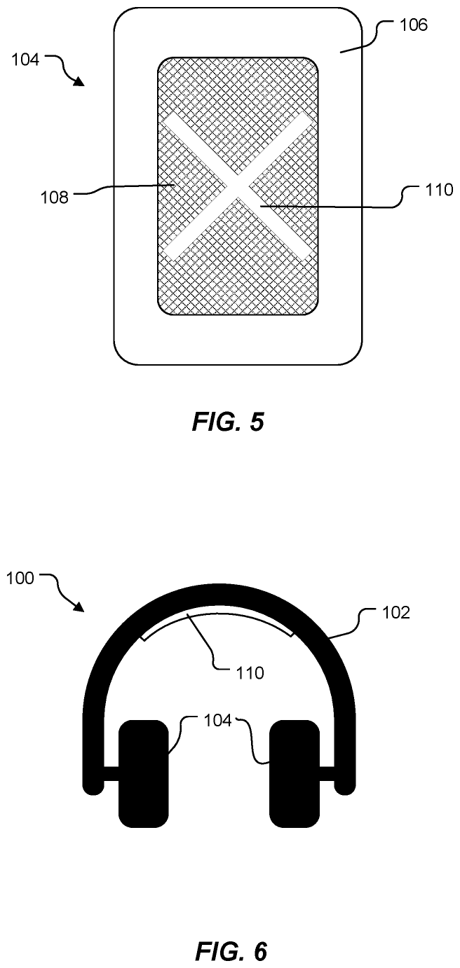

FIG. 5 is a schematic illustration of the inside of an earcup 104 in accordance with disclosed embodiments. As shown UVB light source 110 may be configured in other shapes, such as the exemplary cross pattern shown, and may be incorporated into a front surface of a speaker 108, or the like.

FIG. 6 is a schematic illustration of headgear 100 in accordance with disclosed embodiments. As shown UVB light source 110 may be incorporated into headband 102 instead of, or in addition to, UVB light sources in the earcups 104. Other embodiments are also possible.

Although various embodiments have been shown and described, the present disclosure is not so limited and will be understood to include all such modifications and variations would be apparent to one skilled in the art.

What is claimed is:

1. Headgear to be worn on a wearer's head, the headgear comprising:
an earcup; and
a UVB light source within the earcup.

2. The headgear of claim 1, wherein the UVB light source comprises a light emitting diode (LED).

3. The headgear of claim 1, wherein the UVB light source comprises a strip of light emitting diodes (LEDs).

4. The headgear of claim 3, wherein the earcup further comprises a cushion around a perimeter of the earcup and the strip of LEDs is positioned within a space on an inner surface of the cushion.

5. The headgear of claim 2, wherein the earcup further comprises a space for a wearer's ear within the earcup and the LED is positioned within the space for a wearer's ear.

6. The headgear of claim 5, wherein the UVB light source comprises at least one LED and the UVB light source substantially fills the space for a wearer's ear.

7. The headgear of claim 5, wherein the UVB light source comprises at least one LED and the UVB light source is cross shaped.

8. The headgear of claim 1, wherein the earcup includes a sound emitting source and a cushion and the UVB light source is positioned within a space between the cushion and the sound emitting source.

9. The headgear of claim 1, wherein the earcup includes a sound emitting source and the UVB light source is positioned on a front surface of the sound emitting source.

10. The headgear of claim 9, wherein the UVB light source comprises at least one LED and the UVB light source is cross shaped.

11. Headgear to be worn on a wearer's head, the headgear comprising:
at least one earcup;
a headband to be worn over a wearer's head; and
a UVB light source positioned in the headband.
